SEQUENCE LISTING (6 sequences)

SEQ ID NO: 1 - DNA, 549 bp, Homo sapiens
tggagaccaacacccttcccaccgccactcccccttcctctcagggtccctgttccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaaccccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccgcatggccaaagccgcggcagtcggcatcgacctgggcaccacctactcctgcgtgggggtgttccaa

SEQ ID NO: 2 - DNA, 2103 bp, Homo sapiens
gggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacaaatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagctttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacaccaagctaatttttgtattttttagtagagatggggtttcaccatcttggccaggctggtcttgaattcctgacctcagttgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgtt

SEQ ID NO: 3 - DNA, 20 bp, Artificial Sequence
  Description of Artificial Sequence Primer
accaacacccttcccaccgc

SEQ ID NO: 4 - DNA, 20 bp, Artificial Sequence
  Description of Artificial Sequence Primer
gttatccggaccgcttgccc

SEQ ID NO: 5 - DNA, 25 bp, Artificial Sequence
  Description of Artificial Sequence Primer
ctcgatctcgccgccaactggtaga

SEQ ID NO: 6 - DNA, 22 bp, Artificial Sequence
  Description of Artificial Sequence Primer
ttcggctgggcatggtggttca

(19) United States
(12) Patent Application Publication
(10) Pub. No.: US 2006/0024280 A1
(43) Pub. Date: Feb. 2, 2006

(54) Stem cell-derived endothelial cells modified to disrupt tumor angiogenesis

(75) Inventor: Michael West, Southborough, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Advanced Cell Technology, Inc.

(21) Appl. No.: 11/228,549

(22) Filed: Sep. 16, 2005

Related U.S. Application Data
(63) Continuation of application No. 10/348,359, filed on Jan. 22, 2003, now abandoned.
(60) Provisional application No. 60/349,345, filed on Jan. 22, 2002.

(51) Int. Cl. C12Q 1/68; A61K 48/00

(52) U.S. Cl. 424/93.21; 424/93.7; 435/6

(57) ABSTRACT

The present invention provides cloned, genetically modified, endothelial cells, and the stem cells from which they are derived, which are produced by somatic cell nuclear transfer. The invention further provide novel therapeutic methods in which such cells are administered to a patient with tumors to inhibit and/or disrupt angiogenesis of the tumors, thereby inhibiting tumor growth and killing tumor cells.

### CROSS REFERENCE TO RELATED APPLICATION

This Application claims priority to U.S. Provisional Application Ser. No. 60/349,345filed on Jan. 22, 2002, which is incorporated herein by reference in its entirety.

### FIELD OF THE INVENTION

The present invention provides cloned, genetically modified, endothelial cells, and the stem cells from which they are derived, which are produced by somatic cell nuclear transfer. The invention further provide novel therapeutic methods in which such cells are administered to a patient with tumors to inhibit and/or disrupt angiogenesis of the tumors, thereby inhibiting tumor growth and killing tumor cells.

### BACKGROUND OF THE INVENTION

Angiogenesis is the process by which new blood vessels grow from the endothelium of existing blood vessels in a developed animal; it is an essential for wound healing and for reproduction. Angiogenesis is also rate-limiting step in tumor development. In the absence of the blood supply provided by angiogenesis, tumor growth is limited to 1-2 mm³. Tumors larger than this that are deprived of their blood supply become necrotic and apoptotic (Neithammer et al., 2002, Nature Medicine 8(12):1369). Much attention has focused recently on the notion that tumor growth can be inhibited by blocking or disrupting angiogenesis with agents that target vascular endothelial cell surface proteins or their ligands. (For example, see Folkman,1997, “Angiogenesis and angiogenesis inhibition,” EXS, 79:18; Huang et al., 1997, “Tumor infarction in mice by antibody-directed targeting of tissue factor to tumor vasculature,” 275:547; and Wei et al., 2000, “Immunotherapy of tumors with xenogeneic endothelial cells as a vaccine,” 6:1160-6). Because tumor cells require a blood supply, local interruption of the tumor vasculature produces “an avalanche of tumor cell death” (Huang et al., supra). The strategy of targeting endothelial cells of tumor vasculature is also advantageous because, unlike the tumor cells, vascular endothelial cells are not transformed to have resistance to therapy, and the vascular endothelium is in direct contact with the blood and is relatively accessible to therapeutic agents and cells and factors of the patients immune system (Huang et al., supra). Examples of inhibitors of angiogenesis that are being developed for use as antitumor agents include endostatin and angiostatin, which are naturally occurring angiogenesis inhibitors, and neutralizing antibodies targeted to endothelial cell growth factor reECPtors, such as the Vascular Endothelial Growth Factor Receptors (VEGFR). Specific targets are VEGFR-1 (also known as Flt-1), VEGFR-2 (also known as KDR, Flkl), and VEGFR-3 (also known as Flt4). Current strategies to inhibit angiogenesis by soluble factors suffer from the disadvantage that they typically require frequent (often daily) dosing. The proteinaceous factors cannot be administered orally, so the cost of administration is generally relatively high, and there is a risk of poor compliance. Many of the current strategies of inhibiting tumor angiogenesis through the administration of soluble factors are directed by the model that tumor angiogenesis resulted from the recruitment of neighboring capillary endothelial cells that simply “branched” into the growing tumor mass. However, recent studies suggest that tumor angiogenesis may proceed, at least in part, through a unique and unexpected pathway. Endothelial cell precursors have been shown to circulate in the blood and selectively migrate, or “home,” to sites of active angiogenesis (U.S. Pat. No. 5,980,887 (Isner et al., the contents of which are incorporated herein by reference in their entirety). Circulating bone marrow-derived endothelial cell precursors are also recruited to contribute to angiogenesis by vascularizing tumors. Bone marrow-derived endothelial cells are a major component of the endothelium of a tumor mass, and impairment of the ability to recruit these bone marrow-derived endothelial cells for tumor angiogenesis has been shown to block tumor growth (Lyden et al., 2001, “Impaired recruitment of bone marrow-derived endothelial and hematopoietic precursor cells blocks tumor angiogenesis and growth,” Nature Medicine, 7(11): 1194-1201).

### BRIEF DESCRIPTION OF THE FIGURES

FIG. 1. The nucelotide sequence of the 5′ region of the hsp 70 gene (Genbank accession no. X04676) (SEQ ID NO: 1).

FIG. 2. The nucleotide sequence of the gene encoding Tissue Factor (FIII, Genbank Accession No. XM_{—}001322) (SEQ ID NO: 2).

### DESCRIPTION OF THE INVENTION

Tumor angiogenesis is a process in which endothelial cells are recruited from neighboring, preexisting capillaries and from the circulating blood to form a vascular bed to provide blood to a growing tumor. The present invention provides novel therapeutic methods employing cell therapy to inhibit tumor angiogenesis. The invention provides methods for producing genetically modified hematopoietic stem cells (HSCs) and endothelial cell precursors (ECPs) that give rise to endothelial cells that home to developing tumor vasculature and inhibit or disrupt tumor angiogenesis, resulting in inhibition of tumor growth and in a decrease in tumor mass.

In one embodiment of the present invention, HSCs or ECPs are isolated from a human or a non-human mammal by known methods, and are genetically modified in vitro to contain a genomically integrated DNA expression construct encoding a gene that disrupts or inhibits angiogenesis when it is expressed by endothelial cells in a vascularizing tumor.

In an alternative embodiment of the invention, somatic cells (e.g., fibroblasts or epithelial cells) are isolated from a human or a non-human mammal and are genetically modified to contain a gene that disrupts or inhibits angiogenesis when expressed by endothelial cells in a vascularizing tumor. The genetically modified cells are then cloned by somatic cell nuclear transfer to produce totipotent or pluripotent embryo-derived stem cells (e.g., embryonic stem cells) that can be induced to differentiate into HSCs, which in turn can differentiate to generate genetically modified ECPs of the invention.

The genetically modified HSCs or ECPs, obtained either by direct isolation or by nuclear transfer are then administered to a patient with cancerous tumors; whereupon the HSC-derived ECPs and/or ECP-derived endothelial cells home to sites of tumor angiogenesis and incorporate into the developing vasculature (see, for example, Asahara et al., 1997, “Isolation of putative progenitor endothelial cells for angiogenesis,” Science 275: 964-967, the contents of which are incorporated herein by reference), where they express the gene that causes disruption or inhibition of tumor angiogenesis, and consequently inhibit the growth and reduce the mass of the affected tumors.

Direct Isolation of HSCs and ECPs:

HSCs that differentiate to form ECPs, and ECPs that give rise to endothelial cells, can be isolated directly from bone marrow, fetal liver, circulating peripheral blood, and autologous umbilical cord blood. The leukocyte fraction of peripheral blood is a useful source of ECPs. In addition, ECPs can be produced in vitro or in vivo through the differentiation of HSCs. For example, in addition to giving rise to such cells as B and T lymphocytes, granulocytes, and monocytes, HSCs isolated from adult human bone marrow also differentiate into non-hematopoietic lineages (lin-) that give rise to ECPs that generate cells capable of forming blood vessels in vitro and in vivo (Otani et al., Nature Medicine, 2002,8(9): 1004-1010). Bone-marrow reconstituting HSCs and ECPs both have the CD-34 antigenic determinant (U.S. Pat. No. 5,980,887, supra.) and express vascular endothelial growth factor receptor-1 (VEGFR-1) (Lyden et al., 2001, supra.). ECPs also express the antigenic determinant AC133 (Peichev et al., Blood, 2000, 95(3):952-958); and ECPs and vascularizing endothelial cells both express vascular endothelial growth factor receptor-2 (VEGFR-2) (Neithammer et al., 2002, supra.). Because ECPs are present in circulating blood, they are also referred to as circulating endothelial precursor cells (CEPS) (see U.S. Patent Application No. 60/349,345, the priority of which is claimed, and Lyden et al., 2002, supra). Bone-marrow reconstituting HSCs and ECPs can be isolated from bone marrow, fetal liver, or circulating blood, using known, standard methods such as fluorescence-activated cell sorting (FACS) or immunomagnetic separation (for example, see Peichev et al., supra; and Otani et al., supra, the contents of both of which are incorporated herein by reference in their entirety).

Production of Genetically Modified Stem Cells and ECPs by Nuclear Transfer Cloning:

Advanced Cell Technology, Inc. (the assignee of this application) and other groups have developed methods for transferring the genetic information in the nucleus of a somatic or germ cell from a child or adult into an unfertilized egg cell, and culturing the resulting cell to divide and form a blastocyst embryo having the genotype of the somatic or germ nuclear donor cell. Methods for cloning by such methods are referred to as cloning by “somatic cell nuclear transfer,” because somatic donor cells are commonly used. Methods for cloning by NT are well known, and are described, for example, in U.S. Pat. No. 6,147,276 (Campbell et al.), and in co-owned and co-assigned U.S. Pat. Nos. 5,994,619 and 6,235,969 of Stice et al., the contents of all three of which are incorporated herein by reference in their entirety.

In general, oocytes are isolated from the ovaries or reproductive tract of a human or non-human mammal, matured in vitro, and stripped of cumulus cells to prepare for nuclear transfer. Removal of the endogenous chromosomes of the oocyte is referred to as “enucleation.” Enucleation of the recipient oocyte is performed after the oocyte has attained the metaphase II stage, and can be carried out before or after nuclear transfer. Enucleation can be confirmed by visualizing chromosomal DNA in TL-HEPES medium plus Hoechst 33342 (3 μg/ml; Sigma). Individual donor cells are placed in the perivitelline space of the recipient oocyte, and the oocyte and donor cell are fused together to form a single cell (NT unit) e.g., by electrofusion. The NT units are activated, and are incubated in suitable medium under conditions that promote growth of the NT unit. During this period of incubation, the NT units can be transferred to culture plates containing a confluent feeder layer. Feeder layers of various cell types from various species, e.g., irradiated mouse embryonic fibroblasts, that are suitable for the invention are described, for example, in U.S. Pat. No. 5,945,577, the contents of which are incorporated herein by reference in their entirety. Multicellular non-human NT units produced in this manner can be transferred as embryos into recipient non-human females of the same species as the donor nucleus and recipient oocyte, for development into transgenic non-human mammals. Alternatively, the NT units can be incubated in vitro until they reach the blastocyst stage, and the inner cell mass (ICM) cells of these NT units can be isolated and cultured in the presence or absence of a feeder layer to generate pluripotent or totipotent embryo-derived stem cells, including totipotent ES cells.

Differentiation of ES Cells into HSCs

Methods are known for isolating the ICM cells of a blastocyst produced by NT, and culturing these to generate pluripotent and totipotent embryo-derived cell lines, including totipotent ES cell lines. For example, see co-owned and co-assigned U.S. Pat. Nos. 5,905,042 and 5,994,619 of Stice et al., the contents of both of which are incorporated herein by reference. Using known methods, totipotent and pluripotent stem cells derived from NT-generated blastocysts, e.g., ES cells, can be cultured under conditions that direct or allow differentiation into a variety of partially and fully differentiated somatic cell types, including HSCs. For example, see Wakayama et al., “Differentiation of embryonic stem cell lines generated from adult somatic cells by nuclear transfer, 2001, Science, 292:740-3; Talbot et al., “Spontaneous differentiation of porcine and bovine embryonic stem cells (epiblast) into astrocytes or neurons,” 2002, In Vitro Cell Dev Biol Anim., 38(4):191-7; and Mitalipova et al., “Pluripotency of bovine embryonic cell line derived from precompacting embryos,” 2001, Cloning, 3(2):59-67, the contents of all three of which are incorporated herein by reference. Methods for inducing the differentiation of pluripotent human blastocyst-derived ES cells into HCS cells are also known (U.S. Pat. No. 6,280,718, Kaufman et al., “Hematopoietic differentiation of human pluripotent embryonic stem cells,” the contents of which are incorporated herein by reference).

Cell Therapy Through Therapeutic Cloning:

Methods for human therapeutic cloning have been described. For example, methods that use nuclear transfer cloning to produce cells and tissues for transplant therapies that are histocompatible with the transplant recipient are described in co-owned and co-pending U.S. application Ser. No. 09/797,684 filed Mar. 5, 2001, which also discloses assay methods for determining the immune-compatibility of cells and tissues for transplant the contents of which are incorporated herein by reference in their entirety. Similar methods are also described in U.S. application Ser. No. 10/227,282 (“Screening Assays for Identifying Differentiation-Inducing Agents and Production of Differentiated Cells for Cell Therapy”), filed Aug. 26, 2002, the contents of which are also incorporated herein by reference in their entirety, which further discloses new screening methods that make use of gene trapped cell lines and provide means for efficiently identifying combinations of biological, biochemical, and physical agents or conditions that induce stem cells to differentiate into cell types useful for transplant therapy. Methods for producing totipotent and pluripotent stem cells are also described in co-owned and co-pending U.S. application Ser. No. 09/995,659 filed Nov. 29, 2001, and International Application No. PCT/US02/22857 filed Jul. 18, 2002, which further describe methods for producing histocompatible cells and tissues for transplant by androgenesis and gynogenesis; and in U.S. application Ser. No. 09/520,879 filed Apr. 5, 2000, which discloses methods for producing “rejuvenated” or “hyper-young” cells having increased proliferative potential relative to cells of the donor animal. A method for obtaining totipotent and pluripotent stem cells from embryos generated by parthenogenesis is also reported by Cibelli et al., who describe the isolation of a non-human primate stem cell line from the inner cell mass of parthenogenetic Cynomologous monkey embryos that is capable of differentiating into cell types of all three embryonic germ layers (see Science (2002) 295:819, the contents of which are incorporated herein by reference in their entirety.) The disclosures of all of the above-listed patent applications are also incorporated herein by reference in their entirety.

As an alternative to using nuclear transfer cloning to produce syngeneic ES cells de novo and inducing these to differentiate into the required cells for every patient that is in need of therapeutic transplant, nuclear transfer cloning can be used to prepare a bank of pre-made ES cell lines, each of which is homozygous for at least one MHC gene. The MHC genes, in the case of humans also referred to as HLA (human leukocyte antigen) genes or alleles, are highly polymorphic, and a bank of different ES cell lines that includes an ES cell line that is homozygous for each of the variants of the MHC alleles present in the human population will include a large number of different ES cell lines. Once a bank of such ES cells having homozygous MHC alleles is produced, it will be possible to provide a patient in need of cell transplant with MHC-matched cells and tissues by selecting and expanding a line of ES cells from the ES cell bank that has MHC allele(s) that match one of those of the patient, and inducing the ES cells to differentiate into the type of cells that the patient requires. Methods for preparing a bank of ES cell lines that are homozygous for the MHC alleles, and for using these to provide MHC-matched cells and tissues for transplantation therapies are described in co pending U.S. Provisional Patent Application No. 60/382,616, entitled, “A Bank of Nuclear Transfer-Generated Stem Cells for Transplantation Having Homozygous MHC Alleles, and Methods for Making and Using Such a Stem Cell Bank, filed May 24,2002, the disclosure of which is incorporated herein by reference in its entirety.

The somatic donor cell used for nuclear transfer to produce a nuclear transplant unit or embryo according to the present invention can be of any germ cell or somatic cell type in the body. For example, the donor cell can be a germ cell, or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells. The donor cell can be obtained from any organ or tissue in the body, for example, it can be a cell from an organ selected from the group consisting of liver, stomach, intestines, lung, stomach, intestines, lung, pancreas, cornea, skin, gallbladder, ovary, testes, vasculature, brain, kidneys, urethra, bladder, and heart, or any other organ.

Inhibiting Tumor Angiogenesis Using Genetically Modified Human Cells:

One broad embodiment of the invention comprises isolating human somatic cells, and genetically modifying the cells to contain a gene that disrupts or inhibits angiogenesis when expressed by endothelial cells in a vascularizing tumor. The genetically modified cells are then cloned by somatic cell nuclear transfer as described above, to produce totipotent or pluripotent embryo-derived stem cells (e.g., embryonic stem cells) that can be induced to differentiate into stem cells that give rise to genetically modified endothelial cells that disrupt or inhibit angiogenesis when recruited into a vascularizing tumor. Known methods are used to induce pluripotent embryo-derived stem cells obtained by nuclear transfer to differentiate into cells useful for the present invention, for example, stem cells having a CD34, AC133, VGFR-1, and/or VEGFR-2 surface marker, such as hemangioblasts, HPCs, and ECPs, as discussed above.

Specific genetic modifications of cells of the invention that give rise to human endothelial cells that disrupt or inhibit tumor angiogenesis are discussed below.

Syngeneic Transplant

In a useful embodiment of the invention, somatic cells are taken from a patient with a tumor, genetically modified to contain a gene that disrupts or inhibits angiogenesis, and cloned by somatic cell nuclear transfer to produce pluripotent embryo-derived stem cells that are induced to differentiate into HSCs and ECPs that give rise to genetically modified endothelial cells that disrupt or inhibit angiogenesis when recruited into a vascularizing tumor. The genetically modified HSCs and/or ECPs are then administered to a patient as an autologous transplant, whereupon the HSC- and/or ECP-derived endothelial cells home to sites of tumor angiogenesis, incorporate into the developing vasculature to disrupt or inhibit tumor angiogenesis. Since the transplanted HSCs and ECPs are syngeneic with the patient, they are histocompatible and do not elicit an immune response, unless such a resonse is elicited by expression of the transgene.

An alternative embodiment of the invention that does not use NT-derived cells can be practiced as follows:

ECPs are isolated from the patient, genetically modified in vitro to contain a gene that disrupts or inhibits angiogenesis, and are reintroduced to the patient as described in U.S. Pat. No. 5,980,887 (Isner et al.), the contents of which are incorporated herein by reference in their entirety. In brief, a sample of blood is drawn form the patient, typically 50-200 ml. Prior to venipuncture, the patient can be treated with factors such as Granulocyte Colony Stimulating Factor (GCSF) which stimulates an increase in the number of circulating ECPs. The leukocyte fraction is separated by Ficoll density gradient, then plated briefly to remove adhesive cells. A population of cells positive for antigens specific for ECPs, including but not limited to CD34, VGEFR-2, and AC133, is then isolated. For example, the remaining cells can be treated with fluorochrome labeled antibodies to the antigens specific for ECPs and isolated by Fluorescence Activated Cell Sorting (FACS). Alternatively, ECPs can be isolated by magnetic beads coated with the above antibodies to the above antigens, as is known in the art. Once purified, the population of ECPs are cultured in vitro in suitable medium (e.g., M199 media supplemented with 20% fetal bovine serum), and the cells are genetically modified using methods known in the art. Following genetic modification, the ECPs are intravenously reintroduced to the patient, as described above.

Allogeneic, HLA-Matched Transplant

In another useful embodiment of the invention, the nuclear donor cells that are genetically modified in practicing the invention are not obtained from the patient; rather, they are taken from a person who has HLA alleles that match those of the patent. More simply, the nuclear donor cells are taken from a person who has homozygous HLA alleles that match at least one HLA allele of the patent. A bank of samples of viable nuclear donor cells, each sample made up of cells having homozygous HLA alleles that match an HLA allele found in the population, is prepared and maintained for practicing this embodiment. See co-owned and co-pending U.S. Provisional Patent Application Ser. No. 60/382,616. As described above for syngeneic transplant therapy, genetically modified, HLA-matched HSCs and/or ECPs produced by the invention are administered to a patient as a heterologous transplant, to give rise to endothelial cells that home to and incorporate into the tumor vasculature to disrupt or inhibit tumor angiogenesis. Since the transplanted HSCs and ECPs are HLA-matched to the patient, they are partially histocompatible with the patient, and so do not elicit the strong rejection response that would be elicited by a completely allogeneic transplant.

Allogeneic Transplant

In a third useful embodiment of the invention, allogeneic somatic cells from a person other than the patient are genetically modified to contain a gene that disrupts or inhibits angiogenesis. These cells are then cloned by somatic cell nuclear transfer to produce pluripotent embryo-derived stem cells that differentiate into HSCs and ECPs that give rise to genetically modified endothelial cells that disrupt or inhibit angiogenesis when recruited into a vascularizing tumor. In this embodiment, the genetically modified HSCs and/or ECPs that are transplanted are not HLA-matched to the patient, and they elicit an immune rejection response by the patients immune system that damages the endothelium of the tumor vasculature and contributes to the inhibition of tumor growth.

Results similar to those obtained with the above-described embodiment can also be obtained.

In an alternative embodiment, cells of one or more of the established human ES cell lines are genetically modified, and known methods are used to induce the genetically modified ES stem cells to differentiate into HSCs and ECPs that give rise to genetically modified endothelial cells that disrupt or inhibit angiogenesis when recruited into a vascularizing tumor. Alternatively, HSCs and ECPs can be isolated directly from a person other than the patient and genetically modified to contain a gene that disrupts or inhibits angiogenesis. The genetically modified HSCs and/or ECPs obtained from differentiating ES cells or directly from a person other than the patient can then be transplanted into the patient to disrupt or inhibit tumor angiogenesis, as described above.

Xenogeneic Transplant

In another useful embodiment of the invention, known methods are used to genetically modify somatic cells of a non-human animal so that they contain a stably integrated gene that is expressed in endothelial cells of a vascularizing tumor to disrupt or inhibit angiogenesis. Using known methods, the genetically modified cells are used as nuclear donor cells in a method for cloning by nuclear transfer. The nuclear transfer units obtained by the NT cloning procedure are incubated to produce multicellular embryos, and these are implanted into recipient non-human females of the same species as the donor nucleus and recipient oocyte and allowed to develop into transgenic non-human mammals, as described previously.

Transgenic stem cells such as hemangioblasts, HSCs, and/or ECPs, that give rise to tumor angiogenesis-inhibiting endothelial cells of the invention are isolated from the cloned animals as described. Alternatively, the NT units can be incubated in vitro until they reach the blastocyst stage, and the inner cell mass (ICM) cells of these NT units can be isolated and cultured in the presence or absence of a feeder layer to generate pluripotent or totipotent embryo-derived stem cells, including totipotent ES cells. Known methods are then used to induce differentiation of the NT embryo-derived stem cells into hemangioblasts, HSCs, and/or ECPs, that give rise to the angiogenesis-inhibiting endothelial cells of the invention. The genetically modified stem cells, e.g., HSCs and/or ECPs, isolated from cloned animals or generated by differentiation in vitro, are then administered to a patient with cancerous tumors, whereupon the HSC-derived ECPs and/or ECP-derived endothelial cells home to sites of tumor angiogenesis and incorporate into the developing vasculature, where they effect disruption or inhibition of tumor angiogenesis.

Specific genetic modifications of cells of the invention that give rise to non-human endothelial cells that disrupt or inhibit tumor angiogenesis are discussed below.

The invention can be practiced using cells from any non-human animal species, including but not limited to non-human primate cells, ungulate, canine, feline, lagomorph, rodent, avian, and fish cells. Primate cells with which the invention may be performed include but are not limited to cells of chimpanzees, baboons, cynomolgus monkeys, and any other New or Old World monkeys. Ungulate cells with which the invention may be performed include but are not limited to cells of bovines, porcines, ovines, caprines, equines, buffalo and bison. Rodent cells with which the invention may be performed include but are not limited to mouse, rat, guinea pig, hamster and gerbil cells. Examples of lagomorph species with which the invention may be performed include domesticated rabbits, jack rabbits, hares, cottontails, snowshoe rabbits, and pikas. Chickens (Gallus gallus) are an example of an avian species with which the invention may be performed.

Genetic Modification of Stem Cells and EPCs

Transgenic cells of the invention that are genetically modified to contain a stably integrated gene that is expressed in endothelial cells of a vascularizing tumor to disrupt or inhibit angiogenesis are obtained by routine methods known in the art. Recombinant expression vectors are made and introduced into the cells using standard techniques, e.g., electroporation, lipid-mediated transfection, or calcium-phosphate mediated transfection, and cells containing stably integrated expression constructs are selected or otherwise identified, also using standard techniques known in the art. Methods for making recombinant DNA expression constructs, introducing them into eukaryotic cells, and identifying cells in which the expression construct is stably integrated and efficiently expressed, are described, for example, in Sambrook, et al., Molecular Cloning: A Laboratory Manual, 2d Edition, Cold Spring Harbor Laboratory Press (1989). Such methods useful for practicing the present invention are also described, for example, in U.S. Pat. No. 5,980,887.

A variety of different types of genes that disrupt or inhibit angiogenesis when expressed in endothelial cells of a vascularizing tumor are described below. These genes can be inserted used in the present invention to obtain transgenic stem cells giving rise to endothelial cells that incorporate into tumor vasculature and disrupt or inhibit tumor angiogenesis. The following set of set of angiogenesis-inhibiting genes is intended to be exemplary, and is not intended to be a complete or exhaustive list of the types of genes suitable for the invention. Persons skilled in the art can readily identify and use angiogenesis-inhibiting genes other than those described below to practice the invention. For example, a set of angiogenesis-inhibiting genes that are suitable for use in the present invention that is larger then the set of genes described below is disclosed in U.S. Pat. No. 5,980, 887, and is incorporated herein by reference in its entirety.

Angiogenesis-inhibiting genes such as those described below can be introduced into human or non-human cells to practice the invention in any of its embodiments described herein. Moreover, using the described methods and methods known in the art, persons skilled in the art can stably introduce multiple expression constructs into cells to effect expression of two or more angiogenesis-inhibiting genes into cells of the invention, to enhance the ability of the endothelial cells to disrupt and inhibit tumor angiogenesis.

Increasing Sensitivity of Endothelium to Cytotoxic Stimuli

Using the methods of the present invention, human or non-human cells are genetically modified to produce ECPs that give rise to endothelial cells that cause endothelium in tumor vasculature to have increased sensitivity to radiation, chemotherapy, or other antitumor therapies. For example, the DNA of the cells can be modified to knockout expression of DNA repair genes such as the RAD family of genes, poly (ADP-ribose) polymerase, etc., e.g., by homologous recombination. Alternatively, the human or non-human cells can be genetically modified to produce endothelial cells that have higher propensity to undergo apoptosis upon incurring DNA damage by expressing or increasing the expression of such genes as the ATM, sphingomyelinase, and ceridase genes in the endothelial cells.

Embodiments of the invention in which such cells are used would also comprise administering appropriate treatment to the patient to take advantage of the increased endothelial cell sensitivity and elicit destruction of the sensitized endothelial cells; e.g., radiation or chemotherapy.

Producing a Product Cytotoxic to Vascular Endothelium

The methods of the present invention can be used to obtain human or non-human cells that are genetically modified to produce ECPs that, give rise to endothelial cells that colonize the tumor vasculature and express a cytotoxic product; e.g., ricin.

Inhibiting Tumor Vascularization

Using the methods of the present invention, human or non-human cells are genetically modified to produce ECPs that give rise to endothelial cells that poorly vascularize tumors. For example, homologous recombination en be used to effect the heterozygous knockout of the ldl gene and homozygous knockout of the ld3 genes to produce cells having an (ldl±ld3−/−) genotype.

Inducing Suicide of Vascular Endothelial Cells

The methods of the present invention can be used to obtain human or non-human cells that are genetically modified to produce ECPs that express a selectable suicide gene, such as thymidine kinase (TK), which allows negative selection of grafted cells upon completion of tumor treatment. TK-expressing cells can be negatively selected by the administration of gancyclovir according to methodology known in the art. Alternatively, the cells can be genetically modified to produce ECPs that express cytosine deaminase, which causes the cells to die in the presence of added 5-fluorocytosine. The expressed gene can be lethal as a toxin or lytic agent. A local effect of destroying endothelial cells of tumor vasculature with suicide genes would be the initiation of a cellular responses that block and prevent the of blood into the tumor.

Eliciting Immune Rejection of Vascular Endothelium

Using the methods of the invention, human or non-human cells can be genetically modified to produce ECPs that give rise to endothelial cells that colonize the tumor vasculature and express a cell surface molecule that elicits an immune rejection response. For example, human cells can be genetically modified to produce ECPs that express α 1, 3 galactosyl transferase. This enzyme synthesizes α 1, 3 galactosyl epitopes that are the major xenoantigens, and its expression causes hyperacute immune rejection of the transgenic endothelial cells by preformed circulating antibodies and/or by T cell mediated immune rejection. The gene eliciting immune rejection can be driven by an endothelial-specific promoter, such as the Von Willebrand Factor (vWF). To provide the cells with sufficient time to colonize the tumor, rejection can be delayed by plasmaphoresis, which removes the preformed antibodies from the blood for a period of time. Alternatively, the expression of the rejection-triggering cell surface molecule can be driven by an iinducible promoter, as discussed below.

Diminishing Immune Rejection of Vascular Endothelium

In using the methods of the invention, it may be desirable to genetically modify human or non-human cells to diminish the patient's immune rejection response to the transplanted cells colonizing the tumor vasculature. This can be done by knocking out genes encoding antigenic cell surface proteins that stimulate immune rejection; e.g., T cell receptors human or non-human cells that operate as HLA/MHC antigens, and the α 1, 3 galactosyl transferase gene of non-human mammals.

Stimulating the Patient's Cellular Response to Tissue Damage

The efficacy of the invention can be enhanced by introducing a second genetic modification to produce endothelial cells that also express hyaluronidase, the products of which stimulate the patients cellular response to tissue damage.

Selection of Promoter

In practicing the methods of the invention, promoters are selected that have activities that enhance destruction of the tumors and minimize damage to non-tumor cells and tissues of the patient. The promoters used for the invention can be promoters having constitutive activity in a wide range of cell types, e.g. a viral promoter such as the CMV promoter, or the promoters used can be cell type-specific promoters from genes that are primarily expressed in endothelial cells, e.g., the promoters of the VEGFR-2 and Von Willebrand Factor genes. Promoters of genes such as that are specifically expressed endothelial cells are well known in the art. Since expression of the transgenes in the endothelial cells typically causes cell damage and/or death, it is useful to use and inducible promoter, to be able to control the timing and/or location of expression of the deleterious gene. Promoters inducible by a variety of chemical and physical stimuli are also well known in the art. For example, the hsp 70 promoter is activated by raising the temperature to 43° about 30 minutes (see Example 1). Alternatively, the human EGR-1 promoter is indicibly activated by ionizing radiation (see Joki et al., 1995, Human Gene Therapy, 6:1507-13. One can use an inducible promoter that is not normally active in human cells, and is induced by an exogenous agent to induce expression once the endothelial cells have populated the tumor. For example, constructs having a tetracycline-inducible promoter are commercially available.

Co-Administration of Hematopoietic Stem Cells with the Limited Ability to Form Only Monoclonal or Oligoclonal B and/or T Cells.

Co-owned and co-pending U.S. patent application No. 10/______, entitled “Cloning B and T Lymphocytes”, filed Jan. 15, 2003 (incorporated herein by reference in its entirety), with priority to U.S. Provisional No. 60/348,130 filed Jan. 15, 2002, describes methods using somatic cell nuclear transfor to generate non-human animals that produce monoclonal or oligoclonal B and/or T cells. From such animals, one can isolate hematopoietic stem cells with the limited ability to form only monoclonal or oligoclonal B and/or T cells.

In a useful embodiment of the present invention, the methods of U.S. patent application No. 10/______ are used to produce animals having hematopoietic stem cells with the limited ability to form only monoclonal or oligoclonal B and/or T cells specific for a cell surface antigen that is expressed in the patient by the genetically modified cells in the endothelium of a vascularizing tumor. The targeted surface antigen of the genetically modified cells can be an endothelial cell-specific protein that is normally present on cells of vascular endothelium, e.g., a VEGFR, or it can be a surface antigen that is uniquely expressed in the patient by the genetically modified cells. Transplantation of the genetically modified cells that are recruited into the tumor vasculature as endothelial cells can then be supplemented by also administering hematopoietic stem cells that form monoclonal oligoclonal B and/or T cells specific for the surface antigen of the genetically modified endothelial cells. Alternatively, the genetically modified cells can be transplanted in combination with differentiated monoclonal or oligoclonal B and/or T cells produced by the methods described in U.S. patent application No. 10/______, and specific for the surface antigen of the genetically modified endothelial cells. The genetically modified cells can also be transplanted in combination with administration of a composition comprising monoclonal or oligoclonal antibodies produced by B cells of a cloned animal generated by methods described in U.S. patent application No. 10/______, and specific for the surface antigen of the genetically modified endothelial cells.

Ablation of Endogenous Bone Marrow-Derived HSCs and ECPs

Destruction of tumor vasculature by the methods of the invention may be enhanced by ablating the patients bone marrow prior to administering the genetically modified cells, so that the genetically modified cells form the majority of the pool of stem cells (HSCs and ECPs) giving rise to vascularizing endothelial cells. Ablation of the patients bone marrow can be accomplished by any of the known methods for bone marrow ablation; for example, by radiation, chemotherapy, or with cytotoxic (e.g., radiolabeled) HSC- and ECP-specific antibodies.

Immunoscintiqraphy to Detect Tumor Angiogenesis

In one embodiment, endothelial cells are genetically modified with a recombinant DNA expression construct containing a transgene encoding an antigenic cell surface marker that is not produced by endothelial cells of the transplant recipient. The transgene is under control of a promoter that directs expression of the transgene in endothelial cells participating in tumor angiogenesis. The promoter driving expression of the transgene can be an endothelial cell-specific promoter, e.g., a promoter of an endothelial cell-specific VEGFR gene, or it can be a constitutively active, heterologous promoter such as a CMV promoter. Promoters capable of driving expression of a transgene in vascular endothelial cells are known in the art. Genetically modified endothelial precursor cells (EPCs), or stem cells that differentiate into such EPCs, are transplanted into the patient using known autologous transplant methods.

Once the cells have differentiated and grown into the vascular endothelium of the tumor, immunoscintigraphy using appropriately radiolabeled monoclonal antibodies specific for a marker epitope on the surface of the target endothelial cells can be used to locate vascularizing tumors. Technium 99, Indium 111, and Iodine 131 have been shown to be suitable radiolabels for detection of targeted cancer cells in vivo by the immunoscintigraphy procedure (see Raj et al., 2002, Cancer, 94(4):987-96; and Brouwers et al., 2002, Nucl. Med. Commun., 23(3):229-36).

For example, monoclonal antibodies that have been raised against a cell surface marker epitope that is specifically present on the surface of the target endothelial cells can be labeled with ⁹⁹Tc as described by Schwarz et al. (1987, J Nucl Med; 28:721), the contents of which are incorporated herein by reference in their entirety. The ⁹⁹Tc-labeled monoclonal antibodies are injected intravenously into the patent, and after 10 minutes, the patient is subjected to whole body scintigraphy, for example, using a single head gamma camera equipped with a low energy, parallel-hole collimator as described by Lacic et al. (Nucl Med Comm, 1999; 20:859-865), the contents of which are incorporated herein by reference in their entirety. Analysis of the data collected by the scintigraphic scan allows the practicioner to determine the locations of primary and metastatic tumors undergoing angiogenesis, for later targeting treatment.

### EXAMPLES

### Example 1

### Transplantation and Engrafting of Genetically Modified Endothelial Cells Bovine Model

Primary cultures of bovine fibroblasts are prepared from skin and lung tissue and are grown in vitro using known methods. Such methods are described, for example, in U.S. Pat. No. 6,011,197 (Strelchenko et al.), and in U.S. Pat. No. 5,945,577 (Stice et al.), the contents of both of which are incorporated herein by reference in their entirety.

Fibroblast Isolation

A general procedure for isolating fibroblast cells is as follows: Minced issue is incubated overnight at 10.degree. C. in trypsin EDTA solution (0.05% btypsin/0.02% EDTA; GIBCO, Grand Island, N.Y.). The following day tissue and any disassociated cells are incubated for one hour at 37.degree.C. in prewarmed trypsin EDTA solution (0.05% trypsin/0.02% EDTA; GIBCO, Grand Island, N.Y.) and processed through three consecutive washes and trypsin incubations (one hr). Fibroblast cells are plated in issue culture dishes and cultured in alpha-MEM medium (BioWhittaker, Walkersville, Md.) supplemented with 10% fetal calf serum (FCS) (Hydone, Logen, Utah), penicillin (100 IU/ml) and streptomycin (50 .mu.Vml). The fibroblast cells can be isolated at virtually any time in development, ranging from approximately post embryonic disc stage through adult life of the animal (for example, for bovine, from day 12 to 15 after fertilization to 10 to 15 years of age).

Genetic Modification of Nuclear Transfer Donor Cells

A general procedure for stably introducing a genetic expression construct into the genomic DNA of the cultured fibroblasts by electroporation is described below. Other known transfection methods, such as microinjection or lipofection can also be used to introduce heterologous DNA into the cells.

Culture plates containing propagating fibroblast cells are incubated in trypsin EDTA solution (0.05% trypsin/0.02% EDTA; GIBCO, Grand Island, N.Y.) until the cells are in a single cell suspension. The cells are spun down at 500.times.g and re-suspended at 5 million cells per ml with phosphate buffered saline (PBS). A reporter gene construct containing the cytomegalovirus promoter operably linked to a beta-galactosidase, neomycin phosphotransferase fusion gene (beta-GEO) is added to the cells in the electroporation chamber at 50 .mu.g/ml final concentration. After providing a standard electroporation pulse, the fibroblast cells are transferred back into the growth medium (alpha-MEM medium (BioWhittaker, Walkersville, Md.) supplemented with 10% fetal calf serum (FCS) (Hyclone, Logen, Utah), penicillin (100 IU/ml) and streptomycin (50 .mu.Vml)).

The day after electroporation, attached fibroblast cells are selected for stable integration of the reporter gene by culturing them-for up to 15 days in growth medium containing G418 (400 μg/ml). The neomycin phosphotransferase portion of the beta-GEO gene confers resistance to G418, and cells that do not contain and express the beta-GEO gene are killed by the selection procedure. At the end of the selection period, colonies of stable transgenic cells are present. Each colony is propagated independently of the others. Transgenic fibroblast cells can be stained with X-gal to observe expression of beta-galactosidase, and genomic integration of the expression construct can be confirmed by known methods; e.g., by PCR amplification of the beta-GEO gene and analysis by agarose gel electrophoresis.

Cloning by Nuclear Transfer, Using Transgenic Fibroblasts as Nuclear Donor Cells

Stably transfected fibroblast cells are used as nuclear donors in the nuclear transfer (NT) procedure. Procedures for cloning by NT are well known in the art; for example, methods for cloning by somatic cell nuclear transfer are described in detail in U.S. Pat. No. 6,147,276 (Campbell et al.), and in co-owned and co-assigned U.S. Pat. Nos. 5,945,577 and 6,235,969 of Stice et al.

In general, oocytes are isolated from the ovaries or reproductive tract of a human or non-human mammal and are matured in vitro. The oocytes are stripped of cumulus cells to prepare for nuclear transfer. Enucleation of the recipient oocyte is performed after the oocyte has attained the metaphase II stage, and can be carried out before or after nuclear transfer. For bovine, enucleation can be performed with a beveled micropipette at approximately 18 to 20 hrs post maturation (hpm). Enucleation can be confirmed in TL-HEPES medium plus Hoechst 33342 (3 μg/ml; Sigma). Individual donor cells (fibroblasts) are then placed in the perivitelline space of the recipient oocyte, and the oocyte and donor cell are fused together to form a single cell (an NT unit) using electrofusion techniques; e.g., by applying a single one fusion pulse consisting of 120 V for 15 μsec to the NT unit in a 500 μm gap chamber. For bovine, nuclear transfer and electrofusion can be performed at 24 hpm. The NT units are then incubated in suitable medium; e.g., in CR1 aa medium.

A variety of different procedures for artificially activating oocytes are known and have been described. See co-owned and co-pending U.S. application Ser. No. 09/467,076 (Cibelli et al.), filed Dec. 20, 1999, the contents of which are incorporated herein by reference in their entirety. Following activation, the NT units are washed and cultured under conditions that promote growth of the NT unit to have from 2 to about 400 cells. During this time, the NT units can be transferred to well plates containing a confluent feeder layer, e.g., a feeder layer of mouse embryonic fibroblasts. Feeder layers of various cell types from various species that are suitable for the invention are described, for example, in U.S. Pat. No. 5,945,577. Multicellular non-human NT units produced in this manner can be transferred into recipient non-human females of the same species as the donor nucleus and recipient oocyte, for development into transgenic non-human mammals. Alternatively, the NT units can be incubated until they reach the blastocyst stage, and the inner cell mass (ICM) cells of these NT units can be isolated and cultured in the presence or absence of a feeder layer to generate pluripotent or totipotent embryonic stem cells, as discussed above.

A. Incorporation of Transplanted Cells into the Vascular Endothelium of a Transplant Recipient

Using methods such as those described above, bovine fibroblasts were isolated and stably transfected with a recombinant DNA construct comprising a Neo^{r} gene conferring resistance to neomycin (and G418) under control of a wide-specificity cytomegalovirus (CMV) promoter. The stably transfected fibroblasts were then used as nuclear donor cells and were cloned by nuclear transfer. The transfected fibroblast donor cells were fused with enucleated bovine oocytes to produce NT units that were cultured in vitro to form multicellular embryos, and these were implanted into cows to develop into fetal animals.

The fetal calves were aborted, and fetal liver cells were isolated and injected intravenously into syngeneic adult cows. That is, in each transplant, the cloned, transplanted cells were administered to the same animal from which the donor fibroblasts used to generate the transplanted cells were originally obtained. At 414 days post transplantation, arterial tissue was removed from one of the treated cows (animal # 31) and endothelial cells from the arterial tissue were isolated and expanded. The endothelial cell outgrowths were analyzed to detect cells containing the transgene (Neo^{r}). Of five separate endothelial cell outgrowths, one of them (20%) was positive for the Neo^{r} gene.

Bone marrow stem cells of the cow that received the transplant were isolated and cultured to form primary hematopoietic colonies. Eight pools were made of cells from the primary hematopoietic colonies, each pool consisting of cells from about 40 colonies, and the pools were tested for the presence of cells containing the Neo^{r} transgene. Two of the eight pools tested positive for the Neo^{r} transgene, indicating that approximately 1-2% of the hematopoietic stem cells in the cow's bone marrow were derived from the transplanted transgenic cells. Neo^{r} positive cells were also detected in the lymph nodes of the cow that received the transplant.

These results provide additional evidence that transplanted transgenic, NT-derived hematopoietic stem cells are not rejected by a syngeneic recipient mammal that has an intact and functioning immune system, even though they have heterologous mitochondria. They also demonstrate that the transplanted cells become established in the bone marrow and lymph tissue of the transplant recipient and give rise to differentiated endothelial precursor cells that incorporate into the vascular endothelium of the transplant recipient. The results argue against immune rejection, and in favor of a lower degree of expansion of long-term repopulating stem cells versus short term repopulators.

B. Participation of Transplanted Cells in Neovascularization in a Transplant Recipient

Bovine fibroblasts were isolated and stably transfected with a recombinant DNA construct comprising a Neo^{r} gene under control of a CMV promoter; stably transfected fibroblasts were cloned by nuclear transfer to generate multicellular bovine embryos; and these were implanted into cows to develop into fetal animals, as described in Example 1. Transgenic fetuses were aborted and fetal liver/bone marrow cells were isolated and intravenously injected into an adult cow (animal # 33), also as described in Example 1.

Matrigel (BD) is defrosted overnight in 4° C., and aliquots of 20 ml are mixed with 2 micrograms heparin (Sigma) and 4 micrograms human vascular endothelial growth factor (PeproTech). The Matrigel mixture is injected with pre-cooled syringe subcutaneously at a suitable site. During injection of the Matrigel, the needle is kept in place for approximately 5 min. while lifting up the skin with the needle point, in order to allow the Matrigel to solidify as a plug. After 14-21 days the animal is sacrificed and the Matrigel plugs are removed and cut into two portions. One part of the plug is fixed in 4% paraformaldehyde, embedded in paraffin, sectioned, and H & E stained. Sections are examined by light microscopy, and the number of blood vessels that have formed in the plug is evaluated. The other part of the Matrigel plug is digested by addition of Dispase (Invitrogen) for 5-10 minutes at 37° C. until the gel is liquefied and cells are released. The cells are expanded in vitro and are evaluated to determine their cell type and to detect cells that have a Neo^{r} transgene. Other tissues of the cow, e.g., bone marrow, endothelium, lymph node, etc. are also analyzed to detect and identify cells that have a Neo^{r} transgene.

### Example 2

### Transplantation and Engrafting of Genetically Modified Endothelial Cells-Murine Model

Methods for cloning mice by somatic cell nuclear transfer are known (see Wakayama et al., 1998, “Full-term development of mice from enucleated oocytes injected with cumulus cell nuclei,” Nature 394:369-374, the contents of which are incorporated herein by reference in their entirety). Methods are also known for culturing murine blastocysts produced nuclear transfer to generate an isogenic embryonic stem cell line, for genetically modifying the NT-derived ES cells by homologous recombination, and for inducing the genetically modified ES cells to differentiate in vitro to form hematopoietic precursors that can be therapeutically engrafted into mice in need of the transplant (see Rideout, 3^{rd}, et al., “Correction of a genetic defect by nuclear transplantation and combined cell and gene therapy,” 2002, Cell, 109(1):17-27; the contents of which are incorporated herein by reference in their entirety).

### METHODS

Nuclear Transfer and Embryo Culture

Cloned 129/Sv-ROSA26::LacZ fetuses were produced by piezo-actuated microinjection (Prime Tech, Japan) essentially as described previously (Wakayama et al., 1998, nature 394:369-74; Wakayama and Yanagimachi, 1999, Nature Genetics, 22:127). Nucleus donor cells were isolated from primary cultures derived from tail tip biopsies of 8-week-old 129/Sv-ROSA26::lacZ males and cultured at 37° C. in 5% (vN) CO₂ in humidified air in gelatin-coated 3.5 cm² flasks for 10-14 d in Dulbecco's modified ES medium (DMEM; GIBCO) supplemented with 15% (vN) FCS. Immediately prior to use, cells were dissociated by treating with trypsin and the reaction quenched by the addition of DMEM prior to washing 3× in PBS. A 1-3 ml aliquot of the resultant nucleus donor cell suspension was mixed with a 10-20.ml drop of HEPES-buffered CZB (REF) containing polyvinylpyrrolidone (Mr 360,000) and nuclei injected into enucleated B6D2F1 oocytes within 1 h of mixing. After approx. 1 h, nuclear transfer oocytes were activated by exposure to SrCl₂ for 1 h after which incubation was in KSOM (Specialty Media, N.J.) lacking SrCl2 at 37Â° C. in 5% (vN) C02 in humidified air (Wakayama et al., 1998). Cleaved (2 cell) embryos were transferred the next day (E1.5) to the oviducts of pseudopregnant CD1 surrogate mothers. Cloned fetuses recovered at 11 to 13 days gestation were used as a source of liver cells.

Isolation of c-Kit Positive Liver Cells

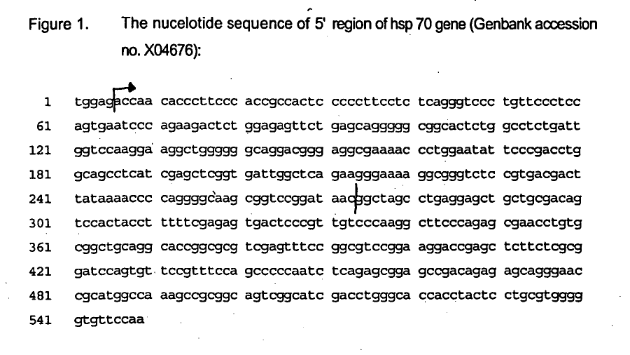

On two separate occasions cloned embryos were obtained, in the first instance a group of four embryos of 12-13 days gestation and in the second, two of 11 and 13 days gestation. Isolated livers were mechanically disaggregated by passage though a 40 micrometer cell strainer (Becton Dickinson, Franklin Lakes, N.J.). A total of 1.67×10⁷ nucleated cells were obtained from the first group, and 5.8×10⁶ from the second. Cells were incubated with PE-conjugated anti-c-kit antibody (BD Pharmingen, San Diego, Calif.), and sorted on a MoFlow cell sorter (Dako Cytomation, Fort Collins, Colo.). In the first study 5×10⁵ c-it+cells were obtained (FIG. 1), and in the second, 1.95×10⁵ c-kit+cells. The cells were suspended in 1 ml phosphate buffered saline with 10% fetal calf serum at 4 C.

The objective of the study was to determine whether fetal liver hematopoietic stem cells (FLHSCs) obtained from cloned fetuses produced by nuclear transfer possess the ability to transdifferentiate and repair damaged tissue (infarcted myocardium) at the site of injection.

Somatic cells were isolated from 1 29/SV EV mice and were genetically modified by insertion of an expression construct directing expression of the LacZ gene into their genomic DNA. The transgenic murine cells were used as nuclear donor cells, and cloned, transgenic fetal mice carrying the LacZ gene were produced by somatic cell nuclear transfer.

Fetal liver hematopoietic stem cells (FLHSCs) were isolated from the cloned fetuses, and a population of c-kit-positive cells was isolated by FACS. Myocardial infarction was induced by occlusion of the left descending coronary artery near its origin in adult 129 SV EV mice. Four to six hours later, approximately 10,000 c-kit-positive FLCs were injected at each of two sites in opposite regions of the border zone, adjacent to the non-contracting dead portion of the left ventricular wall (n=10). Control groups consisted of untreated infarcted mice (n=10) and sham-operated animals (n=9). The three groups of mice were sacrificed one month after surgery or sham operation.

Infarct size was measured by the fraction of myocytes lost by the entire left ventricle inclusive of the interventricular septum. The dimension of the infarct was similar in the two groups of mice exposed to permanent coronary artery ligation. In the treated animals, infarct size was 56±5%, for which the total number of myocytes was 2.72±0.30×10⁶, and the number of myocytes lost was 1.54±0.13×10⁶. In the untreated animals, infarct size was 54±6%, for which the total number of myocytes was 2.72±0.30×10⁶, and the number of myocytes lost was 1.48±0.15×10⁶.

At one month after surgery, the healing process was completed and the area of infarcted myocardium in the untreated mice was a compact scarred area. Analysis of the connective tissue present in the scarred area identified the presence of both collagen type III and collagen type I.

In contrast, myocardial regeneration within the infarct occurred in all mice injected with FLCs. Newly formed myocytes were recognized by the expression of Î±-sarcomeric actin, cardiac myosin heavy chain, connexin 43, and N-cadherin antibody labeling. Importantly, the developed myocardium possessed coronary capillaries, which were identified by factor VIII antibody and Griffonia simplicifolia lectin labeling. Coronary resistance arterioles were numerous and were detected by Î±-smooth muscle actin antibody staining. The arterioles and capillaries contained in their lumen red blood cells, which were stained by TER-119 antibody. The presence of red blood cells in the lumen strongly suggested that the generated vessels were connected with the primary coronary circulation. Labeling with ÃŸ-galactosidase antibody documented that these new structures, including myocytes, endothelial cells and smooth muscle cells, were all ÃŸ-galactosidase-positive and were of FLC origin.

We assayed for production at the site of injection of both myocardium as well as endothelium containing the LacZ gene. Most of the LacZ gene-containing cells that were detected in the repaired tissue were myocardial, but endothelial cells containing the LacZ were detected as well.

Quantitatively, in mice treated with FLCs, the band of regenerated myocardium had an average volume of 7.4±3.0 mm³ and occupied 38±11% of the infarcted scared tissue. Together, 8.2±2.6×10⁶ new myocytes were formed. The volume of these myocytes varied from 200 to 2,700 ¼ m³, averaging 690±160 ¼ m³. There were 250±60 capillaries and 30±10 arterioles per mm² of reconstituted myocardium. The extent of tissue replacement reduced the size of the infarct by 18%, from 56 to 46% of the entire left ventricle. The reduction of infarct size was not sufficient to attenuate the remodeling of the post-infarcted heart Chamber diameter, chamber volume, the wall thickness-to-chamber radius ratio and the left ventricular mass-to-chamber volume ratio were not statistically different from those evaluated in infarcted untreated mice. However, measurements of hemodynamic parameters obtained before sacrifice in the closed-chest preparation showed an improvement of left ventricular end-diastolic pressure in infarcted mice with myocardial regeneration induced by the injection of FLCs. Additionally, diastolic wall stress was reduced by nearly 30% in this group. Thus, FLCs regenerate infarcted myocardium and ameliorate the diastolic properties of the infarcted ventricle.

### Example 3

### Inhibition of Tumors with Endothelial Cells Containing a Construct Directing Heat-Inducible Expression of Human FIII

Porcine somatic cells are isolated, cultured in vitro, and genetically modified using known methods, as described, for example, in co-owned and co-assigned U.S. Pat. No. 6,235,969 (Stice et al.).

A recombinant expression vector is prepared that comprises a gene encoding human tissue factor (FIII) under control of the heat-inducible hsp-70 promoter, and further comprises a Neo^{r} gene conferring neomycin resistance under control of a CMV promoter.

Construction of an Expression Construct Containing the Heat-Inducible hsp-70 Promoter Linked to a DNA Sequence Encoding Human FIII:

(i) The nucleotide sequence of the 5′ promoter region of the hsp 70 gene is shown in FIG. 1 (Genbank accession no. X04676) (SEQ ID NO: 1). PCR primers for cloning a 268 base fragment corresponding to bases 6 to 273 shown in FIG. 1 and containing the functional heat-inducible hsp 70 promoter are shown below.

| | | |
|---|---|---|
| Sense Primer: | ACCAACACCCTTCCCACCGC | (SEQ ID NO:3) |
| Antisense | | |
| Primer: | GTTATCCGGACCGCTTGCCC | (SEQ ID NO:4) |

Human genomic DNA is isolated and a 268 base DNA fragment containing the functional heat-inducible hsp 70 promoter is amplified by standard PCR methods using the primers shown above.

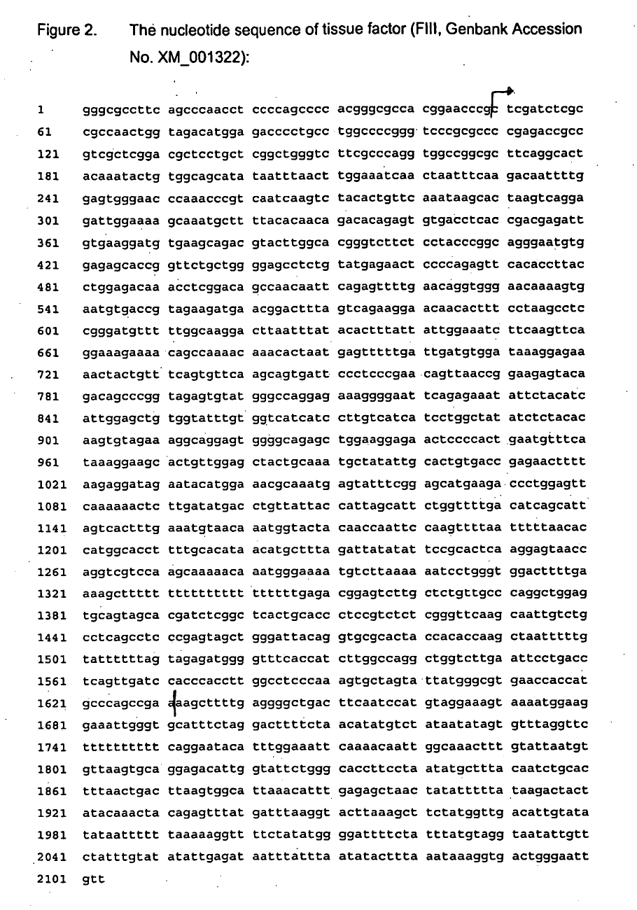

(ii) The nucleotide sequence of the human FIII gene is shown in FIG. 2 (Genbank Accession No. XM_{—}001322) (SEQ ID NO:2). PCR primers for cloning the fragment of the gene corresponding to bases 50 to 1631 shown in FIG. 2 and encoding a functional FIII cell surface protein are shown below:

| | | |
|---|---|---|
| Sense | | |
| Primer: | CTCGATCTCGCCGCCAACTGGTAGA | (SEQ ID NO:5) |
| Antisense | | |
| Primer: | TTCGGCTGGGCATGGTGGTTCA | (SEQ ID NO:6) |

RNA is isolated from human cells expressing FIII, and a DNA fragment encoding a functional FIII polypeptide is amplified by RT-PCR methods using the primers shown above.

(iii) The PCR products are purified, the DNA fragment containing the heat-inducible hsp 70 promoter is ligated to the DNA fragment encoding FIII, and the construct is inserted into a eukaryotic expression vector that also contains an expression cassette containing the selectable Neo^{r} gene under control of a CMV promoter. A linear fragment of the expression vector that comprises both expression constructs is introduced into the porcine cells, and cells in which the construct is stably integrated and the Neo^{r} gene is expressed are selected in medium containing G418.

The genetically modified cells are used as nuclear donor cells to produce cloned, transgenic pigs by nuclear transfer. The nuclear transfer units obtained by the NT cloning procedure are incubated to produce multicellular embryos, and these are implanted into female pigs and allowed to develop into transgenic piglets. Similar methodology is described in Dai et al., 2002, “Targeted disruption of the α1, 3-galactosyltransferase gene in cloned pigs,” Nature Biotechnology, 20:251-255.

Transgenic HSCs and ECPs are isolated from the bone marrow and peripheral blood of the cloned pigs as described. A pharmaceutical composition comprising the genetically modified HSCs and ECPs is administered intravenously to patient with cancerous tumors. A period of 4 to 24 hours is given in which the HSC- and ECP-derived endothelial cells are allowed to form and home to sites of tumor angiogenesis and incorporate into the developing vasculature.

The heat-inducible hsp 70 promoter is activated by locally raising the temperature to 43° C. at sites of tumors in the patients body for 30-40 minutes, using methods known in the art Clinically, heat shock (hyperthermia) can be achieved using x-ray radiation, laser, and MRI with focused ultrasound. Current technology allows various cancers including the ovary (Leopold et al., 1993, Int. J. Radiat. Oncol.Biol. Phys., 27:1245-51), brain (Sneed et al., 1991, Neurosurgery, 28: 206-15), breast (Vernon et al., 1996, Int.J. Radiat. Oncol. Biol. Phys., 35: 73144), prostate (Anscher et al., 1997, Int. J. Radiat Oncol. Biol. Phys., 37:1059-1065) and head and neck (Valdagni et al., 1994, Int. J. Radiat Oncol. Biol. Phys., 28: 163-9), to be heated to a temperature range that is adequate for heat-inducible gene expression (Huang et al., 2000, Cancer Research, 60: 3435-39). Moderate temperatures are needed for effective heat-induced gene expression. After 20 years of experimental cancer treatment with hyperthermia, no long-term side effects have been observed (Huang et al., 2000, supra.).

Heat induction of the hsp 70 promoter leads to strong expression of the FIII gene after about 24 hours (see Veckris, 2000, J. Gene Med., 2:89-96; the contents of which are incorporated herein by reference in their entirety). FIII (tissue factor) is the high-affinity for plasma factors VII and VIIa. These factors bind to the newly produced FIII (tissue factor) molecules on the surfaces of the transgenic endothelial cells that have seeded the tumor vasculature and initiate a blood coagulation cascade that results in the formation of blood dots that occlude the blood vessels of the tumor. Deprived of blood, the tumor cells die, and tumor regression occurs.

In an alternative embodiment, destruction of tumor cells is enhanced by administering a pharmaceutical preparation containing an amount of plasma factors VII and/or VIIa sufficient to enhance coagulation in the tumor vasculature.

In another embodiment, the pigs that are used in the method are α1,3-galactosyltransferase-deficient, to avoid eliciting immune rejection of the transplanted cells and their progeny (see Phelps et al, 2003, “Production of α1,3-galactosyltransferase-deficient pigs,” Science, 299:411-414).

The heat-inducible gene expression system described above can be combined with conventional therapies (radiation and chemotherapy) for enhanced antitumor efficacy.

What is claimed:

1. A method for decreasing tumor mass in a cancer patient, comprising:
(a) ablating bone marrow from said patient, and
(b) grafting endothelial cell precursors (ECPs) or precursors thereof into said patient such that a decrease in tumor mass results, wherein said ECPs or precursors thereof are genetically modified to mediate a decrease in tumor mass.

2. The method of claim 1, wherein said precursors of ECPs are selected from the group consisting of hemangioblasts and bone marrow precursors such as CD34+Acl33+VEGFR2+cells.

3. The method of claim 1, wherein said ECPs or precursors thereof are syngeneic, allogeneic or xenogeneic with respect to said patient.

4. The method of claim 3, wherein said ECPs or precursors are syngeneic, and said ECPs or precursors are produced by nuclear transfer.

5. The method of claim 4, wherein the nuclear donor cell is genetically modified to mediate a decrease in tumor cell mass prior to nuclear transfer.

6. The method of claim 1, wherein said ECPs or precursors are genetically modified such that the resulting endothelium in the tumor vasculature has greater sensitivity to radiation, chemotherapy and/or other tumor therapies.

7. The method of claim 6, wherein said ECPs or precursors contain a genetic knockout of at least one gene involved in DNA repair, wherein said at least one gene is selected from the group consisting of the RAD family of genes and poly (ADP-ribose) polymerase.

8. The method of claim 1, wherein said ECPs or precursors are genetically modified to show increased apoptosis in the presence of DNA damage.

9. The method of claim 8, wherein such modifications occur in a gene selected from the group consisting of the ATM gene, sphingomyelinase and c genes.

10. The method of claim 1, wherein said ECPs or precursors are genetically modified such that they poorly vascularize tumors.

11. The method of claim 10, wherein said genetic modification consists of a heterozygous knockout of the ldl gene and a homozygous knockout of the ld3 genes.

12. The method of claim 1, wherein said ECPs or precursors are genetically modified such that when the cells contact the tumor vasculature, they express at least one toxin or cell surface molecule causing immune-mediated rejection of said ECPs.

13. The method of claim 12, wherein a gene encoding said toxin is operably linked to endothelial specific promoter.

14. The method of claim 2, wherein said toxin is ricin.

15. The method of claim 1, wherein said ECPs or precursors are genetically modified to permit negative selection for said ECPs or precursors in said patent.

16. The method of claim 15, wherein said cells are genetically modified to express thymidine kinase.

17. The method of claim 1, further comprising treating said patient with radiation or chemotherapy simultaneously, previously, or subsequently to said ECP treatment

18. The method of claim 1, further comprising coadministration of hematopoietic stem cells that differentiate into monoclonal or oligoclonal B and/or T cells.
